(19) United States
(12) Patent Application Publication
(10) Pub. No.: US 2012/0064606 A1
(43) Pub. Date: Mar. 15, 2012

(54) METHOD FOR PRODUCING HIGH CONCENTRATE LACTIC ACID BACTERIA WITH MEMBRANE BIOREACTOR AND FREEZE-DRIED, LACTIC ACID BACTERIA POWDER

(75) Inventors: Young Chai Cho, Busan (KR); Il Seong Jung, Seongnam-si (KR); Tae Man Jung, Suwon-si (KR); Sil Ho Choi, Busan (KR); Jin Young Choi, Busan (KR)

(73) Assignee: AMBIO CO., LTD., Busan (KR)

(21) Appl. No.: 13/147,237

(22) PCT Filed: Jul. 7, 2009
(86) PCT No.: PCT/KR2009/003719

§ 371 Date: Oct. 21, 2011

(30) Foreign Application Priority Data
Feb. 2, 2009 (KR) 10-2009-0008033

(51) Int. Cl. C12N 1/20

(52) U.S. Cl. 435/252.9; 435/252.1; 435/253.4

(57) ABSTRACT

The present invention relates to a method for producing lactic acid bacteria of a high concentration continuously using a membrane bioreactor. Specifically, the present invention relates to a method for producing lactic acid bacteria of a high concentration in a membrane bioreactor that comprises the steps of cultivating lactic acid bacteria in a membrane bioreactor including a membrane for product separation and a medium supply apparatus; supplying culture media to the bioreactor through the medium supply apparatus; continuously separating and discharging culture filtrate through the membrane for product separation; and recycling lactic acid bacteria continuously to the bioreactor. Additionally, the present invention relates to a method for producing lactic acid bacteria powder by freeze drying the bacteria produced in the membrane bioreactor using a freeze drying preservative composition. The lactic acid bacteria obtained through the membrane bioreactor is obtained in pellet form with a centrifugal separator, and is subjected to freeze-drying using the composition for freeze drying preservative containing certain amounts of trehalose, maltodextrin, starch and sodium carboxy methyl cellulose to form lactic acid powder. The lactic acid bacteria that have been converted into powder by such method exhibit superior stability chemically and physically compared to lactic acid bacteria powder that has been subjected to simple freeze drying.

### TECHNICAL FIELD

The present invention relates to a method for continuously producing highly-concentrated lactic acid bacteria using a membrane bioreactor. Additionally, the present invention relates to a method for producing lactic acid bacteria powder having superior physical and chemical stability by freeze-drying the bacterial cells obtained in the membrane bioreactor with the addition of a freeze-drying preservative composition.

### BACKGROUND OF THE INVENTION

Lactic acid bacteria have been closely related with the history of mankind and have brought many benefits to human beings. They dwell symbiotically in the gastrointestinal tract to aid digestion and play an important role in the absorption of nutrients. With the interest in health recently increased, the lactic acid bacterium is considered as one of the critical elements for the prevention of disease and for long life, and is designated as a probiotic, which is the concept compared with an antibiotic. Lactic acid bacteria have been used in a variety of products, such as fermented milk, health functional food, beverages and animal feed, since they were first found in yoghurt, and their usage has expanded to a new field of application where antibacterial active materials they produce are advantageous.

Lactic acid bacteria can be cultured in batch mode or continuously. Current research and development is directed to a continuous culturing process, while a batch process was usual and popular previously. However, bacterial cell concentration which can be obtained by either of the processes is very limited, since their metabolites act as an inhibitor of their growth. Lactic acid bacteria metabolize sugars, such as glucose and lactose to produce lactic acid, other organic acids and active materials that kill harmful bacteria in human and animal intestines. However, a large amount of lactic acid, organic acids (e.g., acetic acid), hydroxides which have been produced in the metabolic pathway, and peptides increases hydrogen ion concentration in the culture, thereby the metabolism and growth of lactic acid bacteria are inhibited.

Since the producing method of lactic acid bacteria powder by freeze-drying in a previous batch-processed culture leads to a product still containing organic acids and other metabolites and the freeze-drying is carried out by using a cryoprotectant, cell death ratio during the process and susceptibility to death during distribution of the product are disadvantageously high.

Therefore, to solve the above-mentioned problems, the present inventors have devised a method for producing highly-concentrated lactic acid bacteria, which comprises cultivating lactic acid bacteria in a membrane bioreactor including a membrane for product/metabolite separation and a medium supply apparatus, continuously removing lactic acid and organic acids which inhibit the growth of the bacteria while continuously supplying culture media. The inventors have developed lactic acid bacteria powder having remarkably enhanced stability, which has been produced by freeze-drying the bacterial cell culture having its metabolites removed with the addition of a freeze-drying preservative composition.

### DETAILED DESCRIPTION OF THE INVENTION

### Technical Problems

The purpose of the present invention is to provide a method for producing highly-concentrated lactic acid bacteria using a membrane bioreactor.

Another purpose of the present invention is to provide a method for producing lactic acid bacteria powder having superior physical and chemical stability, which comprises freeze-drying the highly-concentrated bacterial cells produced in the membrane bioreactor using a freeze-drying preservative composition.

### Means for Solving the Problems

To achieve the above-described purpose, the present invention provides a method wherein bacterial metabolites (which act as lactic acid bacteria growth inhibitors), such as lactic acid and organic acids, are removed through the membrane of the bioreactor and culture media is continuously supplied in order to increase biomass. More specifically, the present invention provides a method for producing highly-concentrated lactic acid bacteria, which comprises the steps of cultivating lactic acid bacteria in a membrane bioreactor including a membrane for product/metabolite separation and a medium supply apparatus; supplying culture media to the bioreactor through the medium supply apparatus; continuously separating and discharging culture filtrate through the membrane for product/metabolite separation; and recycling lactic acid bacteria continuously to the bioreactor. To achieve the additional purpose as mentioned above, the present invention provides a method for producing lactic acid bacteria powder, which comprises freeze-drying the highly-concentrated bacteria produced in the membrane bioreactor using a freeze-drying preservative composition.

### Effects of the Invention

Lactic acid bacterial cells can be concentrated by cultivating lactic acid bacteria in a membrane bioreactor including a membrane for product/metabolite separation and a medium supply apparatus, continuously supplying culture media to the bioreactor through the medium supply apparatus, and continuously removing lactic acid and other organic acids which inhibit the growth of the bacteria through the membrane for product/metabolite separation. The method of the present invention is economical compared to previous batch methods in that cost for equipment and operation can be reduced due to a smaller reactor volume requirement and a high yield of cell biomass with respect to culturing time. Further, lactic acid bacteria powder having superior physical and chemical stability can be produced by freeze-drying the concentrated bacterial cells obtained according to the present invention with the addition of a freeze-drying preservative composition.

### BRIEF DESCRIPTION OF THE INVENTION

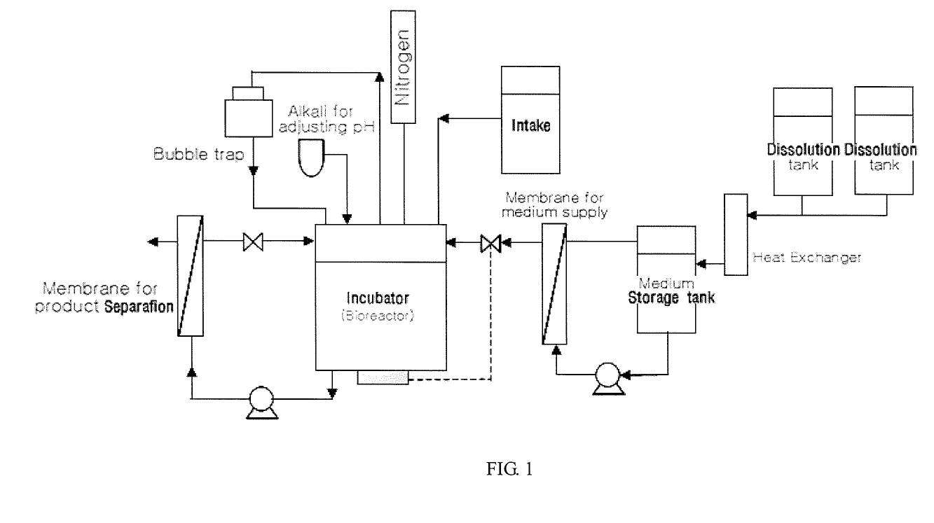

FIG. 1 is a schematic representation of the process of culturing lactic acid bacteria through the membrane bioreactor according to the present invention.

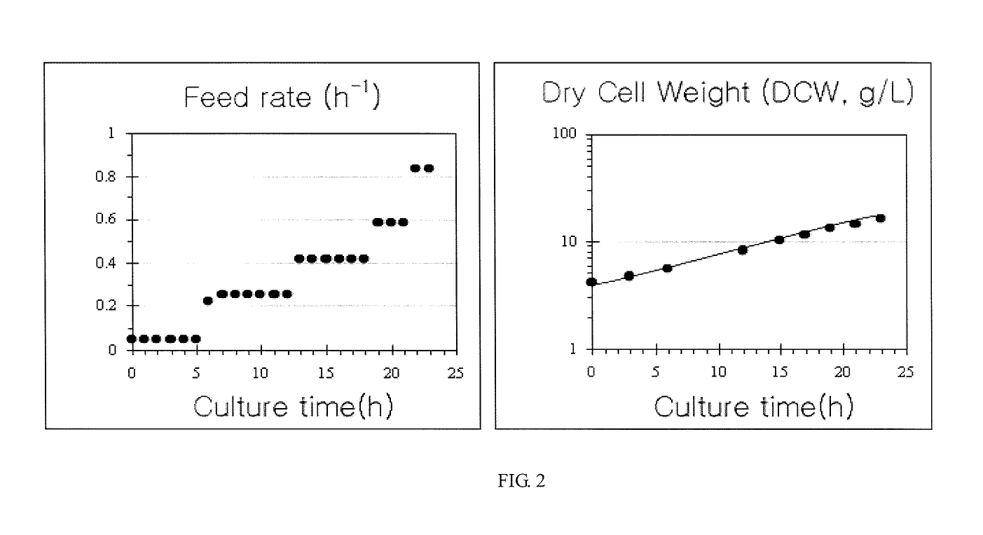

FIG. 2 is a graph showing the feed rate of culture media and the cell biomass obtained, respectively, with respect to the culturing time of a Lactobacillus plantarum strain according to the present invention.

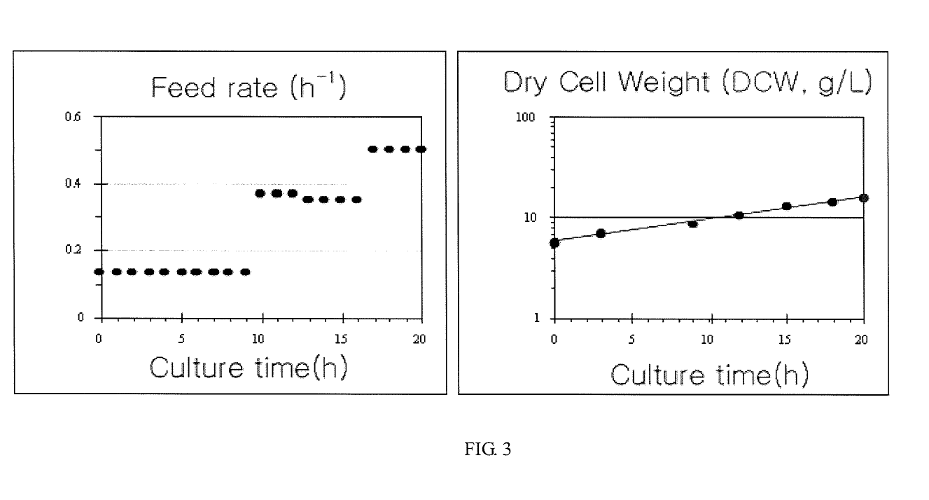

FIG. 3 is a graph showing the feed rate of culture media and the cell biomass obtained, respectively, with respect to the culturing time of a Lactobacillus rhamnosus strain according to the present invention.

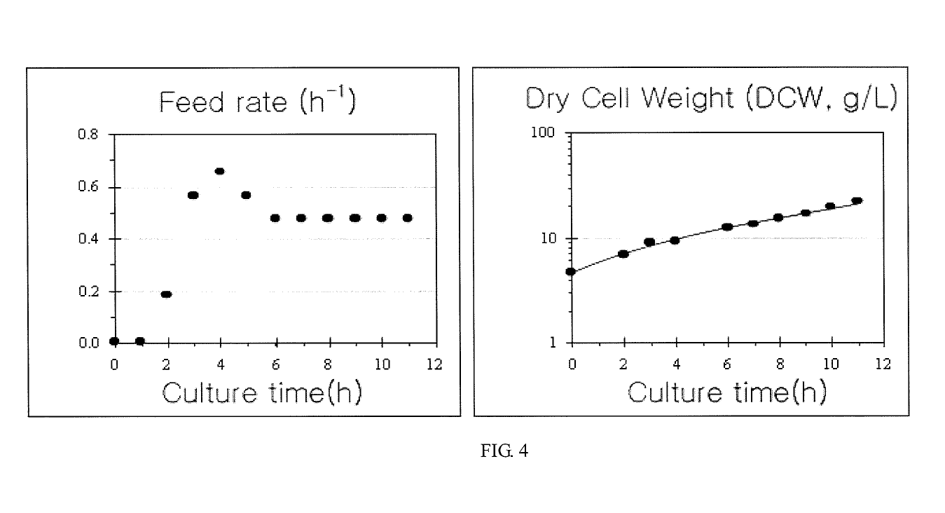

FIG. 4 is a graph showing the feed rate of culture media and the cell biomass obtained, respectively, with respect to the culturing time of a Bifidobacterium longum strain according to the present invention.

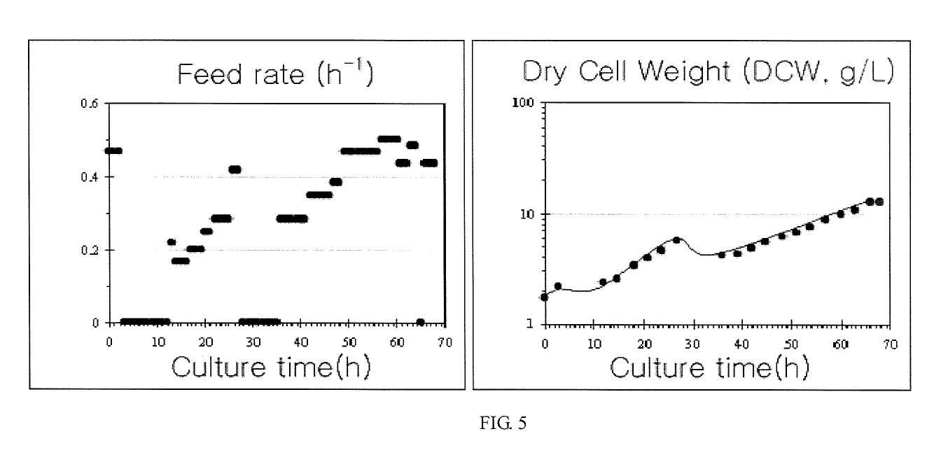

FIG. 5 is a graph showing the feed rate of culture media and the cell biomass obtained, respectively, with respect to the culturing time of a Streptococcus lactis strain according to the present invention.

### MODES FOR CARRYING OUT THE INVENTION

The present invention relates to a method for producing highly-concentrated lactic acid bacteria, which comprises the steps of cultivating lactic acid bacteria in a membrane bioreactor including a membrane for product/metabolite separation and a medium supply apparatus; supplying culture media to the bioreactor through the medium supply apparatus; continuously separating and discharging culture filtrate through the membrane for product/metabolite separation; and recycling lactic acid bacteria continuously to the bioreactor.

Specifically, the membrane bioreactor according to the present invention includes a membrane for product/metabolite separation and a medium supply apparatus. Lactic acid bacteria are about 4 μm in size, and thus, the pore size of the membrane is preferably 0.1 to 4 μm so that lactic acid bacteria fail to pass through the membrane and are recycled to the bioreactor while culture filtrate containing bacterial metabolites, such as lactic acid and other organic acids, is continuously separated and discharged. Nitrogen gas which has been introduced into the bioreactor to create an anaerobic environment, along with carbon dioxide gas derived from the carbon sources in the media, causes bubbling, which interrupts the flow of culture fluid being circulated by a pump. According to the present invention, it is possible to remove such bubbles and foam through the membrane in the bioreactor, which is very important for homogeneous mixture and fluid flow. The medium supply apparatus can be further equipped with a membrane for medium supply, which can be omitted where a highly soluble, separately sterilized medium is used.

The method for producing lactic acid bacteria according to the present invention shows remarkably enhanced productivity for the strains of genus Lactobacillus, Bifidobacterium and Streptococcus.

Additionally, lactic acid bacterial cell powder having remarkably enhanced stability can be produced by freeze-drying the highly concentrated bacterial cell culture obtained in the membrane bioreactor according to the present invention with the addition of a freeze-drying preservative composition. Specifically, the freeze-drying preservative composition is an aqueous solution comprising 5 to 40%, preferably 5 to 20% of trehalose; 5 to 40%, preferably 5 to 20% of maltodextrin; 5 to 19%, preferably 10 to 15% of starch; and 1% of sodium carboxymethylcellulose. The aqueous, freeze-drying preservative solution can further comprise polydextrose or lactose, the concentration of which is preferably 1 to 20%, more preferably 1 to 10% (polydextrose), or preferably 1 to 5% (lactose), respectively. As a component of the freeze-drying preservative composition according to the present invention, trehalose alleviates freezing and/or freeze-drying stress developed during the freeze-drying process. Maltodextrin and polydextrose impart a coating effect to the bacterial cells obtained to prevent external, physical and chemical damage after they are converted into powder form. Lactose and starch block water, and sodium carboxymethylcellulose acts as a thickener to assist the protection of lactic acid bacterial cells by the freeze-drying preservative components. The freeze-drying preservative composition is mixed with the lactic acid bacterial cells obtained in the membrane bioreactor according to the present invention and is freeze-dried to produce lactic acid bacterial cell powder having remarkably enhanced physical and chemical stability.

### EXPERIMENTAL EXAMPLE

Strains, Media and Analysis

Lactobacillus plantarum (KCTC3928), Lactobacillus rhamnosus (KCTC3929), Bifidobacterium longum (KCTC5084), and Streptococcus lactis (ATCC12929) were tested. BL media (Difco) was used for seed culturing of the Bifidobacterium longum strain and MRS media (Difco) was used for the remaining three strains.

Cultivation was scaled up from flask culturing to a pH-controlled jar fermenter, and then to culturing in a membrane bioreactor. The compositions of the culture media used in the examples are as below:

TABLE 1
Composition of culture media for lactic acid bacteria

| | Lactobacillus | Bifidobacterium | Lactobacillus | |
|---|---|---|---|---|
| Strains | plantarum | longum | rhamnosus | Streptococcus lactis |
| Medium | aqueous solution of | aqueous solution of | aqueous solution of | aqueous solution of |
| composition | glucose 3%, | lactose 2.5%, | glucose 3%, | glucose 3%, |
| | soy peptone 0.5%, | soy peptone 1%, | soy peptone 0.5%, | soy peptone 2%, |
| | casein peptone 2%, | casein peptone 1%, | casein peptone 2%, | yeast extract 1.5%, |
| | yeast extract 1%, | yeast extract 1.5%, | yeast extract 1%, | dipotassium |
| | dipotassium | glutamic acid 0.05%, | dipotassium | phosphate 0.05%, |
| | phosphate 0.1%, | Vitamin C 0.05%, | phosphate 0.1%, | ammonium citrate |
| | sodium acetate | dipotassium | sodium acetate 0.1%, | 0.05%, |
| | 0.1%, | phosphate 0.1%, | ammonium citrate | magnesium sulfate |
| | ammonium citrate | sodium carbonate | 0.1%, | 0.01% and |
| | 0.1%, | 0.05%, | magnesium sulfate | manganese sulfate |
| | magnesium sulfate | sodium acetate 0.1%, | 0.01% and | 0.005% |
| | 0.01% and | magnesium sulfate | manganese sulfate | |
| | manganese sulfate | 0.01%, | 0.005% | |
| | 0.005% | manganese sulfate | | |
| | | 0.005% and | | |
| | | iron sulfate 0.001% | | |

In order to check the degree of cell growth, optical density was spectrophotometrically measured. The optical density measured by a spectrophotometer was converted to dry cell weight (g/L) with standard curve between optical density and dry cell weight.

Concentrations of the culture medium and the products obtained during the cultivation were analyzed by performing high performance liquid chromatography (HPLC) and gas chromatography (GC). The HPLC conditions for analyzing the concentrations of sugars, such as glucose, fructose, mannitol, etc, and of organic acids, were: (i) Aminex HPX-87H column (7.8×300 mm), Bio-Rad; (ii) column oven temperature of 50° C.; (iii) UV detector, 210 nm; (iv) mobile phase, 5 mM aqueous sulfuric acid solution; (v) flow rate, 0.6 ml/min

Other organic materials produced during the cultivation were analyzed by GC, and the conditions were: (i) ChromPac Capillary column (silica, 25M×0.32 mm ID, CO-WAX57CB, DF=0.2); (ii) mobile phase, nitrogen gas and air; (iii) Flame Ionization Detector, 220° C.; (iv) inlet temperature, 200° C.; (v) column oven temperature; initial temp. 30° C. (2 mins), final temp. 100° C. (5 mins), temperature increasing rate, 40° C./min.

The Construction of the Membrane Bioreactor

The membrane bioreactor used in the present examples was of 40 L capacity, including 25 L in the bioreactor and 11 L in affiliated lines. The present membrane bioreactor further comprised a heat exchanger, two magnetic pumps, and two membranes for recycling and producing cells. A membrane having 2 m² of filtration area was used for product/metabolite separation and a membrane having 0.2 m² of filtration area was used for medium supply. When a highly soluble and separately sterilized culture media was used, the membrane for medium supply was not used.

### Comparative Example 1

### Flask Culturing

Flask culturing was carried out using media as described in Table 1, adjusted to initial pH of 6 to 6.5, at 120 rpm in an incubator at 37° C. No further pH adjustment was made. The concentration of cell mass and productivity are described in Table 3 below.

### Comparative Example 2

### Culturing in a Stirred Tank Reactor

The four lactic acid bacterial strains were cultured in a pH-adjustable, 3 L jar fermenter. The Lactobacillus plantarum strain showed maximal growth rate of 0.20 h⁻¹, dry cell weight of 1.79 g/L at 30 hours, and productivity of 0.06 at this time. The results were remarkably improved compared to the maximal growth rate of 0.09 h⁻¹ which was obtained in the flask culturing where pH was not adjusted during the cultivation. The Lactobacillus rhamnosus strain showed maximal growth rate of 0.20 h⁻¹, dry cell weight of 2.42 g/L at 36 hours, and productivity of 0.07 at this time. The Bifidobacterium longum strain showed maximal growth rate of 0.28 h⁻¹ and dry cell weight of 4.14 g/L at 11 hours, the highest growth rate obtained in the experiments. The Streptococcus lactis strain showed maximal growth rate of 0.11 h⁻¹ and dry cell weight of 0.58 g/L at 29 hours.

Inhibition of Growth of Lactic Acid Bacteria by the Metabolite

The growth of lactic acid bacteria is inhibited by excessive catabolic metabolite such as lactic acid or acetic acid. In order to maintain a higher cell growth rate, it is very important that minimal inhibition concentration of the organic acids begin to inhibit the bacterial growth. Thus, concentrations of lactic acid and acetic acid which show 50% inhibition of the bacterial growth were measured and described in Table 2. In homo-fermentative fermentation, 1 mole of glucose produces 2 moles of lactic acid. Referring to the results of the Lactobacillus plantarum strain, production of 343 mM of lactic acid infers that 170 mM of glucose has been consumed. 170 mM of glucose corresponds to 3% glucose in the medium. Accordingly, in late stage of the cultivation, the growth of lactic acid bacteria will be rapidly inhibited and dyed. It was also found that more rapid removal is necessary for the Bifidobacterium longum and Streptococcus lactis strains.

TABLE 2
Growth inhibition of lactic acid bacteria by organic acids

| | 50% growth inhibition by organic acids (IC₅₀) | |
|---|---|---|
| | Lactic acid (mM) | Acetic acid (mM) |
| Lactobacillus plantarum | 343 | 1525 |
| Lactobacillus rhamnosus | 458 | 903 |
| Bifidobacterium longum | 179 | 127 |
| Streptococcus lactis | 223 | 784 |

### Example 1

### Culturing in a Membrane Bioreactor

The lactic acid bacterial strains were cultured in the membrane bioreactor. Feed rate of substrate was increased with the increase of cell concentration.

As shown in FIG. 2, the Lactobacillus plantarum strain showed DCW of 16.5 g/L at 24 hours. Feed rate of substrate was stepwise increased from 0.047 h⁻¹ to 0.83 h⁻¹ with the increase of cell concentration.

As shown in FIG. 3, the Lactobacillus rhamnosus strain showed DCW of 15.7 g/L at 20 hours. Feed rate of substrate was stepwise increased from 0.13 h⁻¹ to 0.48 h⁻¹. Feed rate could not be further increased beyond 0.48 h⁻¹ because the by-products caused fouling in the membrane.

As shown in FIG. 4, the Bifidobacterium longum strain showed DCW of 23.5 g/L at 11 hours. Feed rate of substrate was increased to 0.57 h⁻¹, where no fouling was observed.

As shown in FIG. 5, the Streptococcus lactis strain showed DCW of 12.9 g/L at 68 hours. Feed rate of substrate was stepwise increased from 0.17 h⁻¹ to 0.5 h⁻¹.

### Example 2

### Comparison of Total Cell Concentration and Productivity

Table 3 provides comparison of total cell concentration and productivity of the Lactobacillus plantarum, Lactobacillus rhamnosus, Bifidobacterium longum and Streptococcus lactis strains, each of which was cultured in three ways, i.e., in a flask, in a stirred tank and in a membrane bioreactor, respectively.

TABLE 3

| | | Total cell conc. | Productivity |
|---|---|---|---|
| Strains | | (Δ dry cell weight, g/L) | Δ dry cell weight g/L · h⁻¹ |
| Lactobacillus | Flask | 1.12 | 0.021 |
| plantarum | Stirred tank | 1.83 | 0.061 |
| | Membrane bioreactor | 16.2 | 0.704 |
| Lactobacillus | Flask | 2.23 | 0.086 |
| rhamnosus | Stirred tank | 2.33 | 0.065 |
| | Membrane bioreactor | 15.5 | 2.018 |
| Bifidobacterium | Flask | 3.23 | 0.135 |
| longum | Stirred tank | 4.04 | 0.367 |
| | Membrane bioreactor | 22.2 | 2.018 |
| Streptococcus lactis | Flask | 0.91 | 0.019 |
| | Stirred tank | 0.54 | 0.019 |
| | Membrane bioreactor | 12.8 | 0.188 |

For all four strains experimented with, total cell concentration was remarkably improved when the culture was performed in the membrane bioreactor. The Lactobacillus plantarum, Lactobacillus rhamnosus, Bifidobacterium longum and Streptococcus lactis strains showed 15.3, 7.3, 5.7 and 22.2-fold higher total cell concentration, respectively, than those obtained in the flask culture.

The productivity was also considerably improved in the membrane bioreactor culture. The Streptococcus lactis and Lactobacillus rhamnosus strains showed 9.5 and 28.9-fold higher productivity, respectively, than those obtained in the flask culture.

### Example 3

### Preparation of Cell Powder and a Coating Agent

The concentrated cells were isolated and further concentrated in a centrifuge (Model SC-35-06-177) operated at a rotation speed of 6,000 to 15,000 RPM to obtain bacterial cell pellets. An aqueous freeze-drying preservative solution, as in Table 4, was prepared. The bacterial pellets were mixed with the preservative solution in weight ratio of 1:1, frozen at a temperature below −55° C. for 2 days, and then placed under 37° C. freeze-dryer condition into powder form. The compositions of the freeze-drying preservative used in the present example are described in Table 4.

TABLE 4
The composition of freeze-drying preservative

| | Lactobacillus | Bifidobacterium | Lactobacillus | |
|---|---|---|---|---|
| Strains | plantarum | longum | rhamnosus | Streptococcus lactis |
| Compo- | Aqueous solution of | Aqueous solution of | Aqueous solution of | Aqueous solution of |
| sition | trehalose 15%, | trehalose 15%, | trehalose 15%, | trehalose 15%, |
| | maltodextrin 15%, | maltodextrin 15%, | maltodextrin 10%, | maltodextrin 10%, |
| | starch 16% and | starch 16%, and | polydextrose 5%, | lactose 5%, |
| | sodium carboxymethyl | sodium carboxymethyl | starch 16% and | starch 16% and |
| | cellulose 1% | cellulose 1% | sodium carboxymethyl | sodium carboxymethyl |
| | | | cellulose 1% | cellulose 1% |

As can be seen from Tables 5 to 7, the lactic acid bacterial cell powder obtained by freeze-drying with the addition of the preservative composition showed a higher stability, compared to that which was obtained by simply freeze-drying without any preservative composition. Additionally, improvements were found in acid-tolerance and bile acid tolerance, which are required for foods, health functional food, and medicines. As indicated in Table 5, the stability of the cell powder according to the present invention is 5 to 50% superior to that of previous lactic acid bacteria products. The viable cell count was made by culturing the Bifidobacterium strain using BL analytical media in an anaerobic jar at 37° C. for three days. The remaining strains were cultured in MRS analytical media in an anaerobic jar at 37° C. for two days.

TABLE 5
The stability at 25° C.

| | | Strains | | | | | | | |
|---|---|---|---|---|---|---|---|---|---|
| | | Lactobacillus | | Bifidobacterium | | Lactobacillus | | | |
| | | plantarum | | longum | | rhamnosus | | Streptococcus lactis | |
| | | viable | | viable | | viable | | viable | |
| | | cells | | cells | | cells | | cells | |
| | | (cfu/g) | viability | (cfu/g) | viability | (cfu/g) | viability | (cfu/g) | viability |
| preservative | 0 day | 2.5 × 10¹¹ | 100% | 1.4 × 10¹¹ | 100% | 4.3 × 10¹¹ | 100% | 2.2 × 10¹¹ | 100% |
| added | 45 days | 2.4 × 10¹¹ | 96% | 4.5 × 10¹⁰ | 32% | 1.8 × 10¹¹ | 42% | 1.8 × 10¹¹ | 82% |
| | 90 days | 1.7 × 10¹¹ | 68% | 4.4 × 10¹⁰ | 31% | 1.6 × 10¹¹ | 37.2% | 1.2 × 10¹¹ | 55% |
| simple | 0 day | 2.5 × 10¹¹ | 100% | 1.4 × 10¹¹ | 100% | 4.3 × 10¹¹ | 100% | 2.2 × 10¹¹ | 100% |
| freeze- | 45 days | 1.2 × 10¹⁰ | 4.8% | 1.3 × 10⁹ | 0.9% | 6.8 × 10¹⁰ | 16% | 1.4 × 10¹⁰ | 6.4% |
| drying | 90 days | 8.0 × 10⁹ | 3.2% | 1.2 × 10⁸ | 0.1% | 3.2 × 10⁹ | 0.7% | 6.8 × 10⁹ | 3.1% |

The results of the acid tolerance test are presented in Table 6. Artificial gastric juice was made by dissolving 2 g of NaCl and 3.2 g of pepsin in 1 L of distilled water and adjusting to pH 2.1 with HCl. Sample powder (10%) was mixed with the artificial gastric juice and incubated in a shaking incubator at 58 rpm, at 37° C. for 60 minutes. Viable cells were counted with above mentioned method.

TABLE 6
Acid tolerance (pH 2.1 artificial gastric juice 90% + lactic acid bacteria 10%)

| | | Strains | | | | | | | |
|---|---|---|---|---|---|---|---|---|---|
| | | Lactobacillus | | Bifidobacterium | | Lactobacillus | | Streptococcus | |
| | | plantarum | | longum | | rhamnosus | | lactis | |
| Preservative | 0 min | 2.5 × 10¹¹ | 100% | 1.4 × 10¹¹ | 100% | 4.3 × 10¹¹ | 100% | 2.2 × 10¹¹ | 100% |
| Added | 60 mins | 2.05 × 10¹¹ | 82% | 4.2 × 10¹⁰ | 30% | 2.4 × 10¹¹ | 56% | 1.4 × 10¹¹ | 64% |
| simple | 0 min | 2.5 × 10¹¹ | 100% | 1.4 × 10¹¹ | 100% | 4.3 × 10¹¹ | 100% | 2.2 × 10¹¹ | 100% |
| freeze- | 60 mins | 2.1 × 10¹⁰ | 8.2% | 4.5 × 10⁹ | 0.3% | 1.9 × 10¹⁰ | 4.4% | 2.4 × 10¹⁰ | 11% |
| drying | | | | | | | | | |

TABLE 7
Bile-acid tolerance (MRSO (Oxgall + MRS media 1%) 90% + lactic acid bacteria 10%)

| | | Strains | | | | | | | |
|---|---|---|---|---|---|---|---|---|---|
| | | Lactobacillus | | Bifidobacterium | | Lactobacillus | | Streptococcus | |
| | | plantarum | | longum | | rhamnosus | | lactis | |
| Preservative | 0 min | 2.5 × 10¹¹ | 100% | 1.4 × 10¹¹ | 100% | 4.3 × 10¹¹ | 100% | 2.2 × 10¹¹ | 100% |
| Added | 60 mins | 6.3 × 10¹⁰ | 25% | 4.9 × 10⁹ | 3.5% | 5.2 × 10¹⁰ | 12% | 3.3 × 10¹⁰ | 15% |
| simple | 0 min | 2.5 × 10¹¹ | 100% | 1.4 × 10¹¹ | 100% | 4.3 × 10¹¹ | 100% | 2.2 × 10¹¹ | 100% |
| freeze- | 60 mins | 2.4 × 10⁸ | 0.1% | <1 × 10⁷ | — | <1 × 10⁷ | — | 4.4 × 10⁸ | 0.2% |
| drying | | | | | | | | | |

1. A method for producing highly-concentrated lactic acid bacteria in a membrane bioreactor including a membrane for product/metabolite separation and a medium supply apparatus, which comprises the steps of:
cultivating lactic acid bacteria in the membrane bioreactor;
supplying culture media to the bioreactor through the medium supply apparatus; and
continuously separating and discharging culture filtrate through the membrane for product separation while continuously recycling lactic acid bacteria to the bioreactor.

2. The method of claim 1, wherein the lactic acid bacterium is selected from the group consisting of Lactobacillus, Bifidobacterium and Streptococcus.

3. The method of claim 1, wherein the pore diameter of the membrane for product separation is 0.1 to 4 μm.

4. A method for producing lactic acid bacteria powder, which comprises freeze-drying the bacterial cells obtained according to the method of claim 1.

5. A method for producing lactic acid bacteria powder, which comprises freeze-drying the bacterial cells obtained according to the method of claim 1 using an aqueous freeze-drying preservative solution comprising 5 to 40% of trehalose, 5 to 40% of maltodextrin, 5 to 19% of starch and 1% of sodium carboxymethylcellulose.
